(19) United States
(12) Patent Application Publication
(10) Pub. No.: US 2025/0076294 A1
(43) Pub. Date: Mar. 6, 2025

(54) RAPID TEST DEVICE FOR THE DETECTION OF SARS-COV-2 VIRUSES AND THE RELATIVE ANTIBODY PRODUCTION

(71) Applicants: UNIVERSITA' DEGLI STUDI MAGNA GRAECIA DI CATANZARO, CATANZARO (CZ) (IT); POLITECNICO DI TORINO, TORINO (TO) (IT); FONDAZIONE BRUNO KESSLER, Trento (TN) (IT)

(72) Inventors: Natalia MALARA, LAMEZIA TERME (IT); Cristina POTRICH, ROVERETO (IT); Cecilia PEDERZOLLI, POVO (IT); Lorenzo LUNELLI, POVO (IT); Candido Fabrizio PIRRI, CESELLE TORINESE (IT); Enzo Mario DI FABRIZIO, TORINO (IT); Francesca FRASCELLA, RIVOLI (IT); Lucia NAPIONE, VINOVO (IT); Marzia QUAGLIO, TORINO (IT); Alessandro CHIADO', LANZO TORINESE (IT)

(73) Assignees: UNIVERSITA' DEGLI STUDI MAGNA GRAECIA DI CATANZARO, CATANZARO (CZ) (IT); POLITECNICO DI TORINO, TORINO (TO) (IT)

(21) Appl. No.: 18/293,090

(22) PCT Filed: Jul. 25, 2022
(86) PCT No.: PCT/IB2022/056843

§ 371 Date: Jan. 29, 2024

(30) Foreign Application Priority Data
Jul. 27, 2021 (IT) 102021000020042

(51) Int. Cl. G01N 33/543; G01N 33/569; G01N 33/68

(52) U.S. Cl. CPC G01N 33/54388; G01N 33/56983; G01N 33/6854

(57) ABSTRACT

A rapid test device for the detection of SARS-CoV-2 viruses and for the relative antibody production including a substrate of the lateral flow immunochromatographic assay divided into 5 regions: a first reception area to receive the biological sample (“Sample Deposition”); a second area where the first reaction takes place between an analyte and the molecule used for its specific identification (“Conjugation pad”); a third area where the signal amplification uses the addition of the gold nanoparticles (“Signal amplification”); a fourth test area (“Test line”) and a fifth control area (“Control Line”) in which a second and third immunoreaction takes place, providing a visual reading of the test result by a window and two results visible by a colored strip. The sample deposition area comprises a filter having porosity of about 200 nm-1 μm.

### FIELD

The present invention relates to a rapid test device for the detection of SARS-CoV-2 virus and the relative antibody production.

### BACKGROUND

As is known, various problems are being encountered in countering the epidemic generated by the rapid spread of the Sars-CoV2 virus, also due to the lack of technological platforms in which to implement the diagnostic systems, which allow to act throughout the territory, for example, nationally, in a capillary way.

It is now evident that in order to limit the spread of infections, the availability of diagnostic devices capable of supporting mass monitoring through a capillary screening of the population, as fast, reliable and low-cost as possible, would be necessary. In particular, it is essential to screen as many symptomatic and asymptomatic subjects as possible, especially in the health sector, from the police and belonging to categories that must be in close contact with other users.

One of the most suitable technologies for rapid detection is that based on lateral flow immunochromatographic assays (Lateral Flow Test-LFT), used in various fields. Devices based on this technology exploit the high affinity and specificity of antigen-antibody recognition to detect the presence, even at low doses, of the analyte (pathogen) of interest. The selection of molecular probes (e.g., antibodies) and related biochemical reagents represents a crucial aspect for the development and success of LFT devices. Typically, a few microliters of sample are needed to support a measurement and the management of the unidirectional flow of the sample is entrusted to the capillarity exerted by suitable components; consequently, the device is passive or does not require automation or fluid handling systems. Also, as regards the biological elements called to react with the sample, no external operations are necessary as all the reagents are already present on the device. This is usually achieved through immobilization processes by absorption and relative drying implemented during the construction phase. Furthermore, for the reading of the on/off response, no further instrumentation is necessary as it is possible to visually perceive the formation of a colorimetric element that indicates in situ the measurement and relative control of the outcome. This analysis system has been widely used, in particular, in the gynecological field for the evaluation of the presence in the urine of human chorionic gonadotropin, a hormone whose levels increase early in pregnancy. The LTF used in this context detects analyte concentrations above a certain threshold, below which the test is to be considered negative or to be repeated.

In particular, and known solutions for the detection of Sars-Cov2 do not pay particular attention to the selection and strategy of use of the reagents suitable to allow the recognition of the ACE-2 receptor with the ligand protein Spike, as well as to the associated functional tests. In fact, specifically for the detection of SARS-CoV-2, the recently available rapid antigen tests intercept specific portions of the viral proteins using mainly monoclonal or polyclonal antibodies.

### SUMMARY

Briefly, the information obtained with the device object of the invention is a complete evaluation of the state of infectivity together with the immunological condition of the subject examined, unlike that which can be obtained with the devices currently available which is incomplete or partial. (i.e. it concerns the detection of the virus or antibodies).

A first solution known in the field of application of the invention is reported in the patent application CN101283093 which describes a method for the determination of the nucleocapsid protein of the SARS-NP virus using a first antibody and a second antibody, both able to bind specifically to SARS-NP and that they can recognize an epitope present in a region between the 283th nucleotide and the 422nd nucleotide at the N end in the amino acid sequence for SARS-NP.

A second solution reported by patent application CN111398603 describes a test strip for immunochromatography to detect antibodies against the new coronavirus and a method of preparation and application of the test strip.

A third patent application CN107202883 describes a lateral flow immunochromatographic analysis device. The product comprises a sample buffer, an absorber buffer and a liner membrane, in which the sample buffer and the absorption buffer are connected to each other.

However, these systems have the limit of not detecting the SARS-CoV-2 virus and antibody production at the same time.

The purpose of the present invention is to provide a rapid test for the detection of SARS-CoV-2 viruses and for the relative antibody production capable of detecting the immunization achieved in the event of contact with the virus, especially if this contact has occurred in a manner subclinical, i.e. without any clinical symptoms.

A further object of the present invention is to provide a rapid test for the detection of SARS-CoV-2 viruses and for the relative antibody production able to give an ON/OFF response in relation, in particular, to the lowering of the detection threshold as a function of the use of ACE-2 as a molecular probe, the creation of optimal functional biointerfaces and an appropriate signal amplification strategy. ACE-2 represents the physiological molecular bait (i.e., the virus exploits the ACE-2 receptor expressed by the epithelial cells to infect them), probably able to increase the probability of binding with the Spike protein ligand expressed by the virus, thus increasing the sensitivity virus detection. The use of ACE-2 offers the opportunity to recognize the virus regardless of the possible variants of the Spike protein.

Furthermore, the object of the present invention is to lower the detection threshold with an increase in diagnostic sensitivity with respect to other similar or alternative systems, conventionally used in clinical practice.

Furthermore, the test according to the invention offers the opportunity to monitor the subject subjected to vaccination in which immunization is in progress. Vaccination-induced antibody production may be either excessive or low, and these levels will affect the type of immune reaction in the event of a probable subsequent encounter with the virus. The vaccinated subject is protected by an effective antibody production which, with the device presented here, can be monitored over time in a simple and practical way. Furthermore, with the simultaneous analytical assay of the virus, the device presented here is able to inform if the same vaccinated subject has had contact with the pathogen and at the same time if its antibody production is efficient. The invention therefore appears to have characteristics such as to exceed those of the current known systems.

According to the present invention, a rapid test is realized for the detection of SARS-CoV-2 virus and for the relative antibody production, as defined in claim 1.

### BRIEF DESCRIPTION OF THE FIGURES

For a better understanding of the present invention, a preferred embodiment is now described, purely by way of non-limiting example, with reference to the attached drawings, in which:

FIG. 1 shows a three-dimensional schematic view of a rapid test device for the detection of SARS-CoV-2 viruses and for the relative antibody production, according to the invention;

FIG. 2 shows a first form of application of the rapid test device for the detection of SARS-CoV-2 viruses and for the relative antibody production, according to the invention;

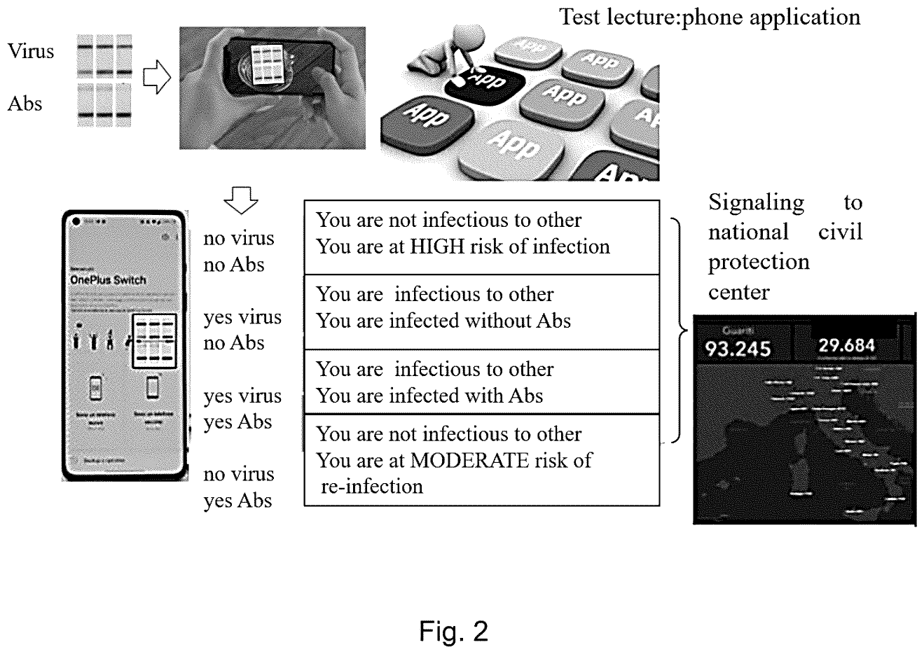

FIG. 3 is representative of the experimental results obtainable using a prototype of the rapid test device for the detection of SARS-CoV-2 viruses and for the relative antibody production, according to the invention.

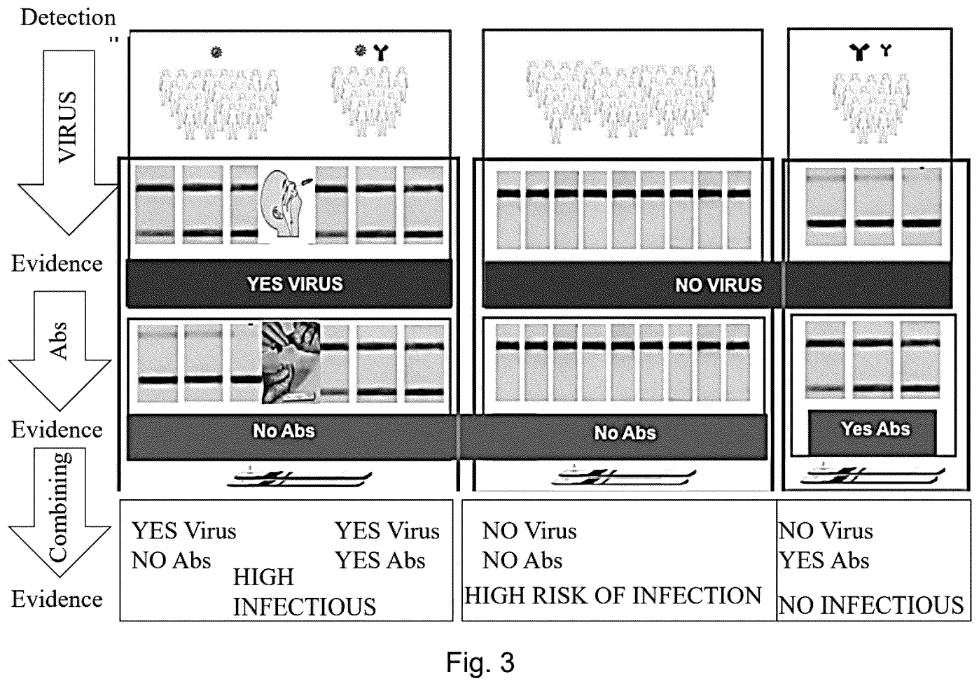

### DETAILED DESCRIPTION

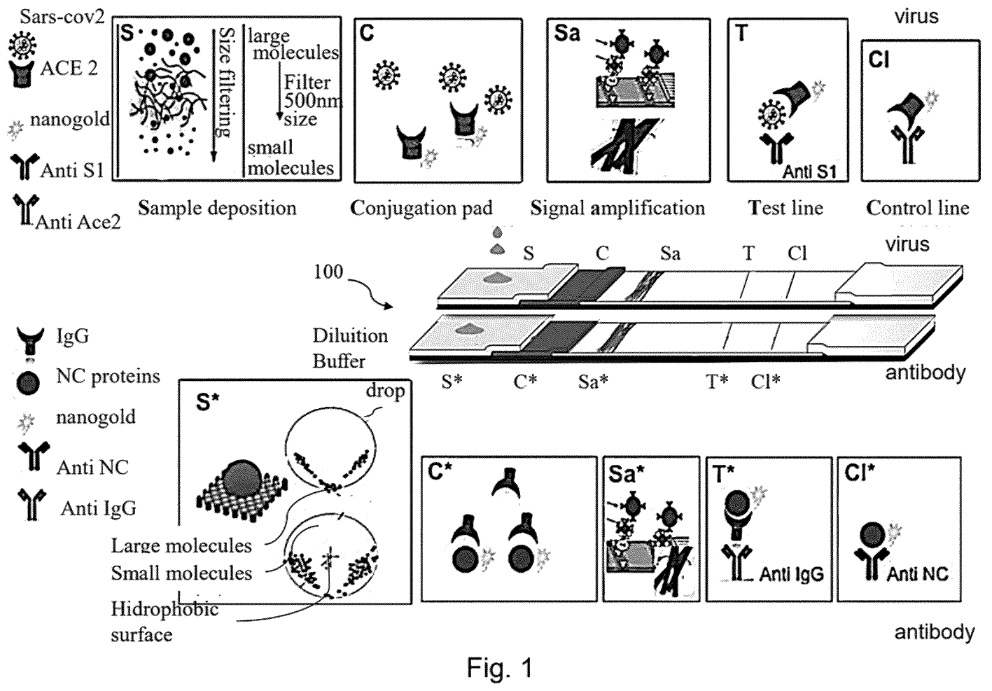

With reference to these figures, and, in particular, to FIG. 1, a rapid test device for the detection of SARS-CoV-2 viruses and for the relative antibody production is shown according to the invention.

The rapid test device 100 for the detection of SARS-CoV-2 viruses and for the relative antibody production comprises a substrate of the lateral flow immunochromatographic assay divided into 5 regions: a first reception area of the biological sample, called “Sample Deposition”; a second area in which the first reaction takes place between an analyte present in the deposited biological matrix and the molecule used for its specific identification, called “Conjugation pad”; a third area, called “Signal amplification”, where the signal amplification strategies are concentrated; a fourth area called “Test line” and a fifth area called “Control Line” in which a second and a third immunoreaction take place aimed at producing the on/off result and its verification.

The device is designed and the technology optimized to allow the visual reading of the test result, possibly with the naked eye, by means of a window and two results visible by means of a colored strip. Viewing the result with the naked eye could create an interpretative flaw in the presence of a double result such as that provided by the combined test. In this regard, the invention envisages being able to be associated with an application based on the image analysis of photographs to offer immediate intelligibility to the user of the test and at the same time to be able to transmit the data, through the mobile telephone system, with after concealing the personal number, in respect of privacy.

According to an aspect of the invention, the “sample deposition” area comprises a filter characterized by a porosity of about 200 nm-1 μm to favor the passage of nanometric particles such as those of the coronavirus and to retain the unwanted biological components.

Advantageously, the filter comprised in the ‘sample deposition area, allows to clean the biological sample retaining unwanted biological components, thereby avoiding possible contamination in subsequent reactions.

According to another aspect of the invention, the “sample deposition” area is structured with raised nitrocellulose to favor a spherical formation of the sample drop diluted with a 1:2 aqueous solution.

Advantageously according to the invention, the virus and the antibodies present in the drop will be transported by the lateral diffusion movement of the flow.

According to an aspect of the invention, the biological matrix used for the evaluation of the virus can be different from the biological matrix used for the evaluation of antibodies, or seroprevalence.

According to one aspect of the invention, the “sample deposition” area is provided differently in the two parallel systems, i.e. one for the antigen (virus) analysis and one for the antibody analysis.

According to another aspect of the invention, for the isolation of the virus the volume of a drop of sample is diluted with aqueous solution which is then deposited on the “Sample deposition” area.

In use, a drop of biological sample is deposited to be examined in the “Sample deposition” area; then the sample flow spontaneously moves from left to right by diffusion gradient. The diffusion coefficient, which is influenced by the structure and density of the biological sample, will influence the degree and rate of diffusion and distribution of the analyte throughout the area of the device, in accordance with Fick's law. In the “Conjugation Pad” area, the recognition takes place between the analyte and the molecule that is specifically responsible for its recognition. For verification, this area is functionalized with ACE2 receptor molecules linked to gold nanoparticles. The corresponding CP (“Conjugation Pad” area) for the evaluation of seroprevalence is functionalized with nucleocapsid proteins (NC) bound to gold nanoparticles. Antibodies directed against virus nucleoproteins are those which have the highest probability of being neutralizing antibodies, i.e. effective in hindering infection.

According to an aspect of the invention, the signal amplification area is common to both LFTs and makes use of the addition of gold nanoparticles of calibrated size and of two different types such as to favor a gradient and a different diffusion of the analyte linked to them in the subsequent areas of the LFT.

According to another aspect of the invention, the “Test line” area in the LFT-virus provides for the presence of antibodies to the Spike protein of the virus, while in the LFT-antibodies the presence of antibodies directed against the nucleoproteins of the viral envelope.

According to one aspect of the invention, the selected molecular probes are immobilized through the formation of covalent bonds with the surface of the materials involved to ensure a suitable conformation, and therefore functionality, of the molecular probes (e.g. antibodies and/or aptamers and/or MIPs (Molecular imprinted polymer)). Furthermore, surface passivation processes will be applied to reduce and control the non-specific adhesion of biomolecules present in the sample that can interfere with the analysis.

According to an aspect of the invention, the starting biological sample is a liquid solution enriched for the presence of exfoliation cells of the oral cavity and of the first respiratory tract, or enriched with intestinal cells or directly a drop of whole blood for the viral antigenic evaluation. For the evaluation of the antibody structure, the biological matrix will be whole blood.

According to another aspect of the invention, the device is based on the different degree of concentration of the virus in a drop of nasal mucus.

According to another aspect of the invention, the device is based on the different concentration degree of the virus in the biological sample considered.

Although the different degree of virus concentration in a drop of blood and in a drop of nasal mucus/saliva suggests as preferential detection of the virus in the nasal mucus/saliva, the device is adaptable to the detection of the virus also starting from blood.

According to an aspect of the invention, modified LFT for the screening of complex diseases can be used by replacing the molecular interlocutors with suitable and specific reagents for the subject disease.

Therefore, the test device for the combined evaluation of the SARS-CoV-2 coronavirus and for the relative antibody production according to the invention is highly specific, fast, low cost, low risk for operators and does not require laboratory equipment.

Another advantage of the test device for the combined evaluation of the SARS-CoV-2 coronavirus and the relative antibody production according to the invention is to be of the ON/OFF type, i.e. to estimate the presence or absence of the SARS-CoV-2 virus and of anti-SARS-CoV-2 antibody production.

Finally, the test device for the combined evaluation of the SARS-CoV-2 coronavirus and the relative antibody production according to the invention is portable and easy to use.

Finally, it is clear that modifications and variations may be made to the test device for the combined evaluation of the SARS-CoV-2 coronavirus and of the relative antibody production according to the invention described and illustrated herein, without departing from the protective scope of the present invention, as defined in the attached claims.

1-10. (canceled)

11. A rapid test device for the detection of SARS-CoV-2 viruses and for the relative antibody production comprising a substrate of the lateral flow immunochromatographic assay divided into 5 regions: a first reception area to receive the biological sample (“Sample Deposition”); a second area where the first reaction takes place between an analyte present in the biological sample and the molecule used for its specific identification (“Conjugation pad”), this second area being functionalized with ACE2 receptor molecules linked to gold nanoparticles and with nucleocapsid proteins (NC) linked to gold nanoparticles, for the detection of the SARS-CoV-2 virus and for the evaluation of seroprevalence, respectively; a third area where the signal amplification uses the addition of the gold nanoparticles of calibrated sizes and of two different types (“Signal amplification”); a fourth test area (“Test line”) and a fifth control area (“Control Line”) in which a second and third immunoreaction takes place comprising, in the portion dedicated to the detection of SARS-CoV-2 viruses, antibodies to the Spike protein of the virus, and in the portion dedicated to the detection of the relative antibody production, the presence of antibodies directed against the nucleoproteins of the viral envelope, providing a visual reading of the test result by a window and two results visible by a colored strip;
wherein the sample deposition area comprises a filter characterized by a porosity of about 200 nm-1 μm to facilitate the passage of nanometric particles such as those of the coronavirus while retaining the unwanted biological components.

12. The rapid test device for the detection of SARS-CoV-2 viruses and for the relative antibody production according to claim 11, being associated with an application based on the analysis of photographs.

13. The rapid test device for the detection of SARS-CoV-2 viruses and for the relative antibody production according to claim 11, wherein the sample deposition area is structured with relief nitrocellulose to favor a spherical formation of the drop of sample diluted with a 1:2 aqueous solution.

14. The rapid test device for the detection of SARS-CoV-2 viruses and for the relative antibody production according to claim 11, wherein the biological matrix used for virus evaluation is different from the biological matrix used for antibody evaluation.

15. The rapid test device for the detection of SARS-CoV-2 viruses and for the relative antibody production according to claim 11, wherein an aqueous solution of 1:2 dilution of a drop of sample is deposited on the “sample deposition area.

16. The rapid test device for the detection of SARS-CoV-2 viruses and for the relative antibody production according to claim 11, wherein the test line area comprises, in the portion dedicated to the detection of SARS-CoV-2 viruses, antibodies to the Spike protein of the virus, and in the portion dedicated to the detection of the relative antibody production, the presence of antibodies directed against the nucleoproteins of the viral envelope.

17. The rapid test device for the detection of SARS-CoV-2 viruses and for the relative antibody production according to claim 11, wherein the main molecular probes (i.e., ACE2 receptor, antibodies and/or aptamers and/MIP (molecular imprinted polymer) directed against the selected viral envelope nucleoproteins are immobilized through the formation of covalent bonds with the surface of the materials involved to ensure a suitable conformation, and therefore functionality, of the molecular probes and wherein surface passivation processes are applied to reduce and control the non-specific adhesion of biomolecules present in the sample capable of interfering with the analysis.

18. The rapid test device for the detection of SARS-CoV-2 viruses and for antibody production according to claim 11, wherein the starting biological sample is a liquid solution enriched for the presence of oral exfoliation cells and of the first respiratory tract, or enriched with cells intestinal or a drop of whole blood directly for viral antigenic evaluation.

19. The rapid test device for the detection of SARS-CoV-2 viruses and for antibody production according to claim 11, wherein it is based on the different degree of concentration of the virus in a drop of nasal mucus.

20. The rapid test device for the detection of SARS-CoV-2 viruses and for antibody production according to claim 11, wherein it is based on the different degree of concentration of the virus in a drop of blood.
